(19) United States
(12) Patent Application Publication
(10) Pub. No.: US 2023/0127982 A1
(43) Pub. Date: Apr. 27, 2023

(54) INDICATOR FILM

(71) Applicant: INSIGNIA TECHNOLOGIES LTD, Lanarkshire (GB)

(72) Inventors: Graham Alexander Skinner, Renfrew (GB); Stuart Russell Cahill Lynch, Glasgow (GB)

(21) Appl. No.: 17/906,397

(22) PCT Filed: Mar. 17, 2021
(86) PCT No.: PCT/GB2021/050668

§ 371 Date: Sep. 15, 2022

(30) Foreign Application Priority Data
Mar. 18, 2020 (GB) 2003925.1

(51) Int. Cl. B32B 27/08; B32B 7/12; B32B 27/36; B32B 27/32; B32B 7/02; B65D 79/02; G01N 31/22; G01N 21/78

(52) U.S. Cl. CPC B32B 27/08; B32B 7/12; B32B 27/36; B32B 27/32; B32B 7/02; B65D 79/02; G01N 31/221; G01N 21/783; B32B 2307/7244; B32B 2307/724; B32B 2307/404; B32B 2255/10; B32B 2307/414; B32B 2553/00; B32B 2439/70; B32B 2307/7246; B32B 2250/24; G01N 2021/775

(57) ABSTRACT

A self-supporting film (10) comprises a gas barrier layer (20); a semi-permeable layer (40); and an indicator material (30), preferably a colorimetric indicator material, is provided between the gas barrier layer (20) and the semi-permeable layer (40). The indicator material (30) is in direct contact with the gas barrier layer (20). The film (10) is particularly useful as an item of packaging, particularly in packaging for perishable materials.

### FIELD OF THE INVENTION

The present invention relates to a self-supporting film, an item of packaging comprising such a film, and use of such a film in packaging, particularly in packaging for perishable materials.

### BACKGROUND

Perishable materials are typically labelled with advice for the consumer as to when such materials should be used or consumed. This is known as an “appropriate durability indication”, or a “date mark” (see Guidance on the application of date labels to food, September 2011, Department for Environment, Food and Rural Affairs). Many perishable materials, such as foods or medicines, are legally required to be labelled by manufacturers with date marks, most frequently “best before” or “use by” dates. However, these dates are calculated based on the assumption that the perishable material is stored under certain conditions, for example under carbon dioxide or refrigerated. A date mark cannot be relied upon when perishable materials have not been stored appropriately, for example, where refrigerators have malfunctioned or are set at a temperature that is higher than those suitable for the perishable material, or where packaging is faulty.

Once the packaging containing a perishable material has been opened, the rate of degradation of the perishable material depends on the conditions to which it is exposed. In addition, degradation may be delayed by storing the perishable material at lower temperatures. In either case, it is useful to determine the length of time that packaging containing a perishable material has been opened.

Colorimetric indicators are a well-known means of detecting the presence of a chemical substance in a particular medium. This type of indicator typically includes pH indicators, which exhibit a colour change as the pH of the medium in which it is placed varies.

Such indicators rely on the optical properties of reactive dyes or inks. These dyes can exist in at least two different chemical states, with each form of the dye absorbing light in a particular range of wavelength. When such a reactive dye existing in a first form is exposed to a given substance, it reacts with the substance via a reversible chemical reaction, thereby turning into a second form of the dye. As the second form of the dye absorbs light at a different wavelength, the chemical reaction provides a colour change, which is visible by an observer.

The use of colorimetric indicators potentially provides an attractive solution to the problem of detecting the presence of some particular chemical substances. Such substances include gases, such as carbon dioxide, oxygen and ammonia, which have particular significance in, amongst other things, food packaging.

Detection of carbon dioxide has always had significance due to the negative effect of carbon dioxide on health if held in too high concentrations. In medicine, carbon dioxide is one of the key, basic analytes routinely monitored in the blood of hospital patients. Capnography is an area in medicine wholly devoted to the monitoring of levels of carbon dioxide in breath. Not only does the presence of carbon dioxide provide important valued medical information, but also its temporal variations in the exhaled breath is used routinely to provide diagnostic information via capnography. In anaesthesiology, one method to ensure the correct placement of the tube carrying the gases to the lungs into the trachea, rather than the oesophagus, is to monitor the level of carbon dioxide (typically 4-5% in exhaled breath).

In the food industry, the use of modified atmosphere packaging (MAP) is well established. MAP packaging involves flushing food with an oxygen-free gas, usually carbon dioxide, and sealing, ready for distribution to the wholesale and/or retail trader. The purpose of MAP packaging is to prevent aerobic spoilage microbe growth, and usually allows food to stay fresh 3-4 times longer. Detection of levels of carbon dioxide in MAP-packaged food is essential to indicate the freshness of the food.

Ammonia (NH₃) is a caustic, hazardous gas with a pungent characteristic odour. It is widely used both directly and indirectly in the production of explosives, fertilisers, pharmaceuticals, household cleaning products and as an industrial coolant. Ammonia and other volatile amines also give spoiled fish its ‘off’ taste and smell, as these are produced as fish meat decays. As a result there is a need to monitor ammonia levels not only in industry to monitor for leaks and waste water effluents, but also in the food packing industry, in particular for fish packaging. After fish are caught and killed micro-organisms form on the skin and scales. These are known as specific spoilage organisms (SSO) which produce ammonia and volatile amines including trimethylamine (TMA) and dimethlyamine (DMA) from the amino acids present in the fish. These microbial degradation products are collectively known as total volatile basic nitrogen (TVB-N). By measuring the TVB-N if would be possible to give a measure of how fresh the fish is.

The main cause of most food spoilage is oxygen, because its presence allows a myriad of aerobic food-spoiling micro-organisms to grow and thrive. Oxygen also spoils many foods through enzyme-catalysed reactions, as in the browning of fruit and vegetables, destruction of ascorbic acid and the oxidation of a wide range of flavours. Many oxidative food-spoiling reactions, including lipid oxidation, occur non-enzymically.

A number of colorimetric indicators capable of detecting the presence of particular analytes have been reported in the literature. Polymer-based compositions incorporating colorimetric indicators, which compositions may be prepared and processed via known polymer processing techniques while maintaining the efficacy and stability of the indicators, are described in GB 2 474 571 A (Mills et al). The polymer composites comprises at least one thermoplastic polymer and at least one chemical indicator.

A multilayer adhesive tape or sticker comprising such colorimetric indicators is described in U.S. Ser. No. 10/107,760 B2 (Smyth et al). The multilayer adhesive tapes or stickers may be attached to the inside of packaging in order to detect oxidising agents, oxygen, water, reducing agents, UV light, carbon dioxide, amines, ammonia, temperature and/or the passage of time. Incorporating the colorimetric indicator into a self-supporting film is not disclosed.

A carbon dioxide sensing colour changeable dye comprising a carbon dioxide status indicator, a solvent, and a polymer in which the carbon dioxide status indicator is dispersed is described in U.S. Ser. No. 14/292,246 (G. Heacock). The dye can be used to form an indicator strip, which can then be placed onto a package.

It is at an object of the present invention to address or mitigate at least one disadvantage associated with the prior art. For example, it is an object to decrease the costs and/or complexity of packaging material and/or of the packaging process.

### SUMMARY

The present invention is based on the finding that an indicator material can be incorporated into a self-supporting film. The resulting self-supporting film may be suitable for use with standard packaging machinery used in typical packaging processes. Consequently, one aspect of the invention provides a self-supporting film comprising an indicator material that may be used in packaging without requiring additional packaging steps, thus potentially reducing the complexity or cost to the packaging process.

According to a first aspect, there is provided a self-supporting film comprising:

(i) a gas barrier layer;

(ii) a semi-permeable layer; and

(iii) an indicator material positioned between the gas barrier layer and the semi-permeable layer;

wherein the gas barrier layer and semi-permeable layer are of substantially equal surface area.

According to a second aspect, there is provided a self-supporting film comprising:

(i) a gas barrier layer;
(ii) a semi-permeable layer; and
(iii) an indicator material provided between the gas barrier layer and the semi-permeable layer;

wherein the indicator material is in direct contact with the gas barrier layer.

According to a third aspect, there is provided a self-supporting film comprising:

(i) an upper or outer gas barrier layer;
(ii) a lower or inner semi-permeable layer;
(iii) an indicator material provided on an inner side of the upper or outer gas barrier layer; and
(iv) an adhesive layer provided between the indicator material and the lower or inner semi-permeable layer;

wherein the upper or outer gas barrier layer and lower or inner semi-permeable layer are of substantially equal surface area.

According to a fourth aspect, there is provided a self-supporting film comprising:

(i) an upper or outer gas barrier layer;
(ii) a lower or inner semi-permeable layer;
(iii) an indicator material provided on an inner side of the upper or outer gas barrier layer; and
(iv) an adhesive layer provided between the indicator material and the lower or inner semi-permeable layer;

wherein the indicator material is in direct contact with the upper or outer gas barrier layer.

According to a fifth aspect, there is provided an item of packaging comprising the self-supporting film according to any one of the first to fourth aspects.

According to a sixth aspect, there is provided use of the self-supporting film of any one of the first to fourth aspects, in an item of packaging.

According to a seventh aspect, there is provided a method of manufacturing a self-supporting film, the method comprising:
(i) providing a gas barrier layer;
(ii) providing a semi-permeable layer;
(iii) applying an indicator material onto the gas barrier layer or the semi-permeable layer; and
(iv) bonding and/or laminating the gas barrier layer and the semi-permeable layer;
wherein the indicator material is positioned between the gas barrier layer and the semi-permeable layer.

Further aspects and embodiments will be evident from the discussion that follows below.

In the discussion that follows, reference is made to a number of terms, which have the meanings provided below, unless a context indicates to the contrary. The nomenclature used herein for defining compounds is in general based on the rules of the IUPAC organisation for chemical compounds, specifically the “IUPAC Compendium of Chemical Terminology (Gold Book)”. For the avoidance of doubt, if a rule of the IUPAC organisation is in conflict with a definition provided herein, the definition is to prevail.

The term “comprising” or variants thereof is to be understood herein to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps.

The term “about” used herein, when qualifying a number or value, is used to refer to values that may lie outside the strictly specified value, for example may lie within ±5% of the value specified. For example, if a semi-permeable layer has an oxygen transmission rate from about 30 to about 200 cc/m²/day, oxygen transmission rates of 28.5 to 210 cc/m²/day may be included.

As summarised above, the first and second aspects are based on the surprising finding that an indicator material can be incorporated into a self-supporting film comprising a gas barrier layer and a semi-permeable layer, wherein the gas barrier layer and semi-permeable layer are of substantially equal surface area and/or the indicator material is in direct contact with the barrier layer. Advantageously, the resultant self-supporting film may be able to indicate a change in the atmospheric conditions of the environment below the gas barrier layer. The semi-permeable layer may allow certain materials or substances, e.g. gases, to flow to and from the indicator material, which may be able to detect and signal a change in atmospheric conditions.

The term “self-supporting” when used in connection with a film is used to refer to a film, sheet or the like that is able to function as an indicator without reliance on further materials, e.g. a supporting layer or substrate.

In accordance with the first to fourth aspects, the self-supporting film comprises a gas barrier layer and a semi-permeable layer, with an indicator material positioned in between the two. The gas barrier layer may act to provide an acceptable physical barrier to a first substance. Whether a physical barrier is acceptable or not for a specific application may depend on the concentration of the first substance permitted through the gas barrier layer, i.e. the permeability of the gas barrier layer to the first substance.

The permeability of a substance is used herein to refer to the ability of a porous material to allow gases to pass through it. High permeability refers to a rapid flow of gas through the material, whilst low permeability refers to a slow flow of gas through the material. The flow of gas through a material is commonly measured as the gas transmission rate, given in units of cc/m²/day, or g/m²/day for water vapour. Unless specified otherwise, the gas transmission rates herein are the values at 25° C. and at a Relative Humidity of 50% (90% for water vapour transmission rates). A high transmission rate of gas through a material corresponds to a high permeability of the material to the gas, and vice versa.

In some embodiments, the gas barrier layer of the first to fourth aspects may have a permeability that is low enough to provide an acceptable barrier to a first substance, i.e. the transmission rate of the first substance through the gas barrier is acceptably low.

Acceptable gas transmission rate values depend on the identity of the first substance and the purpose of the self-supporting film. For example, if the self-supporting film is used to package a highly sensitive perishable material stored under a first substance that is an inert gas, a low transmission rate of the inert gas through the gas barrier layer may be preferred. However, if the self-supporting film is used to package a less sensitive perishable material, then a higher transmission rate of the inert gas may be acceptable. Often, the perishable material may degrade on exposure to oxygen and/or water. Thus, the gas barrier layer may advantageously provide an acceptable barrier to oxygen and/or water vapour, i.e. the oxygen and/or water transmission rates may be suitably low, irrespective of the identity of the first substance.

If the first substance is carbon dioxide then, in order to provide an acceptable barrier to carbon dioxide, the gas barrier layer may typically have a carbon dioxide transmission rate of ≤about 50 cc/m²/day, more typically ≤about 40 cc/m²/day, sometimes ≤about 20, e.g., about 15 cc/m²/day, e.g. ≤about 10 cc/m²/day, preferably ≤about 5 cc/m²/day.

The gas barrier layer may have a carbon dioxide transmission rate from about 0.01 to about 50 cc/m²/day, about 0.01 to about 20 cc/m²/day, about 0.05 to about 15 cc/m²/day, about 0.1 to about 10 cc/m²/day, or about 0.15 to about 5 cc/m²/day. If the first substance is carbon dioxide, then the gas barrier layer may typically have a carbon dioxide transmission rate of about 0.15 to about 5 cc/m²/day, e.g. about 3 cc/m²/day.

If the first substance is oxygen then, in order to provide an acceptable barrier to oxygen, the gas barrier layer will typically have an oxygen transmission rate of ≤about 50 cc/m²/day, e.g. ≤about 40 cc/m²/day, e.g. ≤about 20 cc/m²/day, e.g. ≤about 10 cc/m²/day, preferably ≤about 5 cc/m²/day.

The gas barrier layer may have an oxygen transmission rate from about 0.01 to about 50 cc/m²/day, about 0.01 to about 40 cc/m²/day, about 0.05 to about 20 cc/m²/day, about 0.1 to about 10 cc/m²/day, or about 0.1 to about 5 cc/m²/day. If the first substance is oxygen, then the gas barrier layer will typically have an oxygen transmission rate of about 0.1 to about 5 cc/m²/day, e.g. about 2 cc/m²/day.

If the first substance is water then, in order to provide an acceptable barrier to water, the gas barrier layer will typically have a water vapour transmission rate of ≤about 30 g/m²/day, typically ≤about 20 g/m²/day, e.g. ≤about 10 g/m²/day, e.g. ≤about 5 g/m²/day.

The gas barrier layer may have a water vapour transmission rate from about 0.01 to about 30 g/m²/day, about 0.01 to about 20 g/m²/day, about 0.05 to about 10 g/m²/day, or about 0.1 to about 5 cc/m²/day. If the first substance is water, then the gas barrier layer may typically have a water vapour transmission rate of about 0.1 to about 5 g/m²/day.

The gas barrier layer of the self-supporting film may comprise any one or a combination of materials with a permeability that is low enough to provide an acceptable barrier to a/the first substance. The any one or a combination of materials may provide a gas barrier layer with any one of the gas transmission rates disclosed herein.

The gas barrier layer may typically be transparent or semi-transparent. By such provision, in use, a user or on-looker may be able to view the indicator material through the barrier layer.

The gas barrier layer may comprise, may consist of or may be made of a polymeric material, e.g., a plastic material. The gas barrier layer may comprise any one or more selected from the group consisting of polyethylene terephthalate, polyester, polypropylene, polyethylene, ethylene vinyl alcohol, polyvinylidene chloride and polyvinyl alcohol. Conveniently, the barrier layer may comprise only one of these materials. Typically, the gas barrier layer may comprise or may be made of polyethylene terephthalate. The gas barrier layer may comprise a polyethylene terephthalate layer. Typically, the gas barrier layer may be provided as, may form or may comprise an upper or outermost layer of the film.

As described previously, the self-supporting film comprises a gas barrier layer and a semi-permeable layer, with an indicator material positioned in between the two. In some embodiments, the semi-permeable layer may have a permeability that allows for a controlled flow of a first substance. In these embodiments, the first substance may be able to flow to or from the indicator material via the semi-permeable layer. By “controlled flow” is meant that the semi-permeable layer may be able to delay the flow of the first substance, thereby delaying the change in status of the indicator material. For example, if the indicator material is a colorimetric indicator material that changes colour in the presence of a greater concentration of a first substance, then the semi-permeable layer acts to delay the rate of the colour change. The semi-permeable layer may achieve this by controlling the flow rate of the first substance to or from the indicator material.

If the first substance is carbon dioxide, then, in order to provide a controlled flow of carbon dioxide, the semi-permeable layer may typically have a carbon dioxide transmission rate of ≤about 4000 cc/m²/day, more typically ≤about 1000 cc/m²/day, e.g. ≤about 150 cc/m²/day, e.g. ≤about 125 cc/m²/day, e.g. ≤about 100 cc/m²/day, e.g. ≤about 80 cc/m²/day, e.g. about 40 cc/m²/day.

The semi-permeable layer may have a carbon dioxide transmission rate from about 5 to about 200 cc/m²/day, about 5 to about 160 cc/m²/day, about 10 to about 150 cc/m²/day, or about 10 to about 40 cc/m²/day. If the first substance is carbon dioxide, then the semi-permeable layer may typically have a carbon dioxide transmission rate of about 10 to about 40 cc/m²/day, e.g. about 20 cc/m²/day.

If the first substance is oxygen, then, in order to provide a controlled flow of oxygen, the semi-permeable layer may typically have an oxygen transmission rate of 5 about 4000 cc/m²/day, e.g. ≤about 1000 cc/m²/day, e.g. ≤about 150 cc/m²/day, e.g. ≤about 125 cc/m²/day, e.g. ≤about 100 cc/m²/day, e.g. ≤about 80 cc/m²/day.

The semi-permeable layer may have an oxygen transmission rate from about 30 to about 200 cc/m²/day, about 35 to about 160 cc/m²/day, about 40 to about 150 cc/m²/day, or about 45 to about 80 cc/m²/day. If the first substance is oxygen, then the semi-permeable layer will typically have an oxygen transmission rate of about 45 to about 80 cc/m²/day, e.g. about 60 cc/m²/day.

If the first substance is water then, in order to provide a controlled flow of water, the semi-permeable layer may typically have a water vapour transmission rate of ≤about 50 g/m²/day, e.g. ≤about 40 g/m²/day, e.g. ≤about 30 g/m²/day, e.g. ≤about 20 g/m²/day.

The semi-permeable layer may have a water vapour transmission rate from about 0.01 to about 50 g/m²/day, about 0.1 to about 40 g/m²/day, about 1 to about 30 g/m²/day, or about 5 to about 20 cc/m²/day. If the first substance is water, then the semi-permeable layer will typically have a water vapour transmission rate of about 5 to about 20 g/m²/day.

The semi-permeable layer of the self-supporting film may comprise any one or a combination of materials with a permeability that allows for a controlled flow of the first substance. The any one or a combination of materials may provide a semi-permeable layer with any one of the gas transmission rates disclosed herein.

The semi-permeable layer of the self-supporting film may comprise, may consist of or may be made of a polymeric material, e.g., a plastic material. The semi-permeable layer may comprise any one or more selected from the group consisting of polyester (e.g. polyethylene terephthalate) and polypropylene. Often, the semi-permeable layer comprises only one of these materials. Typically, the semi-permeable layer may comprise or may be made of polyethylene terephthalate. In some embodiments, the semi-permeable layer may be made of, may consist essentially of or may consist of polyethylene terephthalate.

Typically, the semi-permeable layer may be provided as, may form or may comprise a lower or innermost layer of the film.

It will be understood that the gas barrier layer and the semi-permeable layer may have the oxygen, carbon dioxide, ammonia, and water transmission rates described above irrespective of the identity of the first substance. For example, when the first substance is carbon dioxide, the gas barrier layer and semi-permeable layer may have any of the carbon dioxide, oxygen, ammonia and water vapour transmission rates discussed above.

Typically, the gas barrier layer of the self-supporting film may be less permeable to a/the first substance than the semi-permeable layer. The gas barrier layer may be at least 50%, 60%, 70% or 80% less permeable to a/the first substance than the semi-permeable layer. The gas barrier layer may have a carbon dioxide transmission rate of about 0.15 to about 5 cc/m²/day and an oxygen transmission rate of about 0.1 to about 5 cc/m²/day, whilst the semi-permeable layer may have a carbon dioxide transmission rate of about 10 to about 40 cc/m²/day and an oxygen transmission rate of about 45 to about 80 cc/m²/day. In such instance, the permeability of the semi-permeable layer may allow for a controlled flow of a first substance, whilst the gas barrier layer may act to provide an acceptable physical barrier to the first substance. The first substance may be trapped below the gas barrier layer, but may be able to permeate the semi-permeable layer at a controlled or predetermined flow rate, and thus may be able to flow to and from the indicator material. This means that, if the atmosphere below the semi-permeable layer changes, then the first substance may flow to or from the indicator material, changing the concentration of the first substance at the indicator material. The indicator material may then sense a change in the concentration of the first substance, and signal as such.

The gas barrier layer and semi-permeable layer may be of substantially equal surface area. By “substantially equal” is meant that the surface area of the gas barrier layer and that of the semi-permeable layer are generally similar, for example within ±5% of each other. The gas barrier layer and the semi-permeable layer may typically each span substantially the entire surface area of the self-supporting film. For example, the gas barrier layer and semi-permeable layer typically may each extend across at least 95% of the surface area of the self-supporting film, preferably at least 99% of the film. Typically, the indicator material may span a smaller surface area than the gas barrier layer and the semi-permeable layer. The indicator material may be located in discrete sections of the self-supported film.

Although the surface areas of the gas barrier layer and the semi-permeable layer may typically be substantially equal, the depth/thickness of the gas barrier layer and that of the semi-permeable layer may differ.

The flow rates of gases through materials may typically depend on the thickness of the material: the gas transmission rate through a material may be lower when the material is thicker.

The barrier layer of the self-supporting film may typically have a thickness of about 1 to about 100 μm, e.g. about 5 to about 50 μm, e.g. about 5 to about 25 μm.

The semi-permeable layer of the self-supporting film may typically have a thickness of about 1 μm to about 100 μm, e.g. about 5 to about 50 μm, e.g. about 10 to about 35 μm.

If the barrier layer and semi-permeable layer are of the same chemical composition, then the barrier layer may typically be thicker than the semi-permeable layer.

The permeation of gas through the semi-permeable layer may be controlled by altering the thickness of the semi-permeable layer.

In accordance with the first to fourth aspects, the self-supporting film comprises an indicator material positioned between the gas barrier layer and the semi-permeable layer. As described above, the indicator material may be able to detect and signal a change in atmospheric conditions.

The indicator material may be sensitive to the concentration of a/the first substance, i.e. the indicator material may be capable of detecting a change in concentration of a/the first substance.

The indicator material may be or may comprise a colorimetric or luminescence-based indicator material. The wavelength of light absorbed or emitted by the indicator material may be dependent on the concentration of the first substance. The indicator material may typically be a colorimetric indicator.

The indicator material may comprise a substance, e.g. a dye, capable of changing colour when exposed to a first substance. The indicator material may typically comprise a reactive dye or ink. A reactive dye or ink may exist in at least two different chemical states, with each form of the dye absorbing light in a particular range of wavelength. When such dyes are exposed to a first substance, they can reversibly or irreversibly react from a first chemical state into a second chemical state, thereby inducing a visible colour change.

The rate at which the indicator material, e.g. dye, changes from the first chemical state to the second chemical state may depend on the rate at which the concentration of the first substance changes. This in turn may depend on the first substance transmission rate of the semi-permeable layer: a greater first substance transmission rate may lead to a quicker change of the dye from the first to the second chemical state. Thus, the rate of change of the dye from the first to the second state may be controlled by selecting a semi-permeable layer of a specific chemical composition and thickness.

The concentration of the first substance at the indicator material may depend on the amount of time that passes from either the initial exposure of the indicator material to the first substance, or from the initial loss in concentration of the first substance at the indicator material. Therefore, the signal, such as the colour, of the indicator material may relate to the time since its exposure to or reduced exposure to the first substance. Consequently, the indicator material may be used to indicate the amount of time that has passed from its exposure to or reduced exposure to the first material. This may be useful in determining the amount of time that has passed since exposure of a perishable material to a first substance or reduced exposure of a perishable material to a first substance.

Beneficially, the indicator material may have a long storage stability under dark, but otherwise ambient conditions. For example, the indicator material may be stable under these conditions for at least one week, preferably at least one month, more preferably at least six months, and most preferably at least twelve months. A definition of “stable” may be that at least about 95% of the indicator material is retained, i.e. that 5 about 5% of the indicator material has degraded.

The indicator material, e.g. dye, may be sensitive to the temperature at which it is stored, and may be capable of signalling a prolonged change in its storage temperature. For example, the indicator material may be a first colour if kept in a typical domestic freezer at temperatures of less than about −20° C., but a second colour when stored at higher temperatures for a prolonged period. Consequently, the indicator material may be useful in indicating when perishable materials have been subject to an increase in temperature, such as when perishable materials have been defrosted and re-frozen.

Typically, the indicator material may be sensitive to the presence of at least a first substance.

Typically, the first substance to be detected may be a chemical species capable of causing a chemical change in the indicator material, e.g. dye. In other words, the first substance may typically be capable of changing the dye from a first chemical state to a second chemical state.

The first substance may be present in the air and may be a gaseous species such as carbon dioxide, oxygen, ammonia or water vapour. Alternatively, the first substance may be a particulate material or may be in solution or suspension, for example in water. The first substance may be a liquid such as an alcohol, solvent or the like. Preferably, the first substance is carbon dioxide, oxygen, water or ammonia. Most preferably, the first substance is carbon dioxide.

The indicator material, e.g. dye, thereof, may typically be in equilibrium between a first and a second chemical state. The indicator material, e.g. dye, is typically a first colour in the first chemical state, and a second colour in the second chemical state. Preferably, the first and second colours may be different.

In some embodiments, the indicator material may comprise a carbon dioxide-sensitive reactive dye, such as m-Cresol Purple (MCP, m-cresonsulfonphthalein), Thymolphthalein (3,3-bis(4-hydroxy-2-methyl-5-propan-2-ylphenyl)-2-benzofuran-1-one), o-Cresolphthalein (3,3-Bis(4-hydroxy-3-methylphenyl)-1(3H)-isobenzofuranone), Acrylolyoxy florescein (AcFI), β-methyl umbelliferon (BMUB), Bromothymol blue (BTB, 4,4′-(1,1-Dioxido-3H-2,1-benzoxathiole-3,3-diyl)bis(2-bromo-6-isopropyl-3-methylphenol)), 5′ and 6′-Carboxyseminaphtholfluorescein (c-SNAFL), 5′ and 6′-Carboxyseminaphtholrhodamine (c-SNARF), Cresol Red (CR, o-Cresolsulfonephthalein), 2-(2,4-Dinitrophenylazo)-1-naphthol-3,6-disulphonic acid (DNPA), tris(thenoyltrifluoroacetonato) europium (III) ([Eu(tta)₃]), Fluorescein (FI, resorcinolphthalein), 7-hydroxycoumarin-4-acetic acid (HCA), 8-Hydroxypyrene-1,3,6-trisulphonic acid (HPTS), Neutral red (NR, toluylene red), Phenol Red (PR, phenolsulfonphthalein), Rhodamine 6G (R6G), Sulforhodamine 101 (SRh), Thymol blue (TB, thymolsulphonephthalein), and Texas Red hydrazine (THR). It is to be understood that any other pH-sensitive dye or ink may be suitable for use as a CO₂-sensitive reactive dye.

Preferably, the indicator material, e.g. dye, may comprise any one selected from the group containing Cresol Red, m-Cresol Purple, Phenol Red and Thymol blue.

If the indicator material comprises a carbon dioxide-sensitive dye, then the sensitivity of the indicator material to concentration changes of carbon dioxide depends on the pKa of the indicator material. If the indicator material has a higher pKa, such as Thymol blue (which has a pKa of 8.9), then the indicator material has a higher affinity for the protons produced by carbon dioxide on dissolution of the carbon dioxide into a protic solvent, i.e. the indicator material will signal a change in carbon dioxide concentration when the concentration has changed by a smaller amount. In other words, if the indicator material has a higher pKa, it has a greater sensitivity to a change in carbon dioxide concentration. On the other hand, if the indicator material has a lower pKa, such as Phenol red (which has a pKa of 7.6), then the indicator material has a lower affinity for the protons produced by carbon dioxide on dissolution of the carbon dioxide into a protic solvent. This means that indicator materials with lower pKa values will signal a change in carbon dioxide concentration when the concentration has changed by a larger amount. In other words, if the indicator material has a lower pKa, it is less sensitive to a change in carbon dioxide concentration. Therefore, if a signal change is desirable for smaller changes in carbon dioxide concentrations then an indicator material with a higher pKa may be preferred, and if a signal change is desirable for greater changes in carbon dioxide concentrations then an indicator material with a lower pKa may be preferred. For illustrative purposes only, the pKa values of the preferred indicator materials are (in order of highest to lowest) Thymol blue (8.9), m-Cresol purple (8.32), Cresol red (8.2), and Phenol Red (7.6).

In some embodiments, the indicator material may comprise an ammonia-sensitive reactive dye such as Bromophenol Blue (BPB, 4,4′-(1,1-dioxido-3H-2,1-benzoxathiole-3,3-diyl)bis(2,6-dibromophenol)), Bromocresol Green (BCG, 2,6-Dibromo-4-[7-(3,5-dibromo-4-hydroxy-2-methyl-phenyl)-9,9-dioxo-8-oxa-9A6-thiabicyclo[4.3.0]nona-1,3,5-trien-7-yl]-3-methyl-phenol), Bromocresol Purple (BCP, 4,4′-(1,1-Dioxido-3H-2,1-benzoxathiole-3,3-diyl)-bis(2-bromo-6-methylphenol)), Bromothymol Blue), Phloxine Blue (PB, Disodium 2′,4′,5′,7′-tetrabromo-4,5,6,7-tetrachloro-3-oxospiro[2-benzofuran-1,9′-xanthene]-3′,6′-diolate), Thymol Blue, or m-Cresol Purple.

In some embodiments, the indicator material may comprise an oxygen-sensitive reactive dye such as Methylene blue (MB, methylthioninium chloride), Thionine (Th, 3,7-Diaminophenothiazin-5-ium), Azure B (AzB, N,N,N′-Trimethylthionin), Nile blue (NR, [9-(diethylamino)benzo[a]phenoxazin-5-ylidene]azanium sulfate), or any other dye which, upon reduction, is rendered oxygen-sensitive. Reduction of the dye may be effected photochemically, using a semiconductor photocatalyst such as titania, or chemically using a reducing agent such as ascorbic acid. The oxygen-sensitive reactive dye may exhibit fluorescence that is quenched by oxygen. Examples of such dyes include Ruthenium tris bypyridyl (Rubpp), tris(4,7-diphenyl-1,10-phenanthroline) ruthenium (II) perchlorate (Rudpp), Platinum (II) octaethyl porphyrin ketone (PtOEPK), Proflavin (Pf).

In some embodiments, the self-supporting film may comprise more than one indicator material, e.g. more than one dye. By such provision, the self-supporting film may be capable of detecting the presence of more than one type of first substance and/or be capable of detecting changes in the concentration of more than one type of first substance.

If the indicator material comprises a reactive dye, it may be a water-based dye, i.e. the reactive dye may be dissolved or suspended in a water solvent. The indicator material comprises a protic solvent, such as water and/or an alcohol, such as a denatured alcohol, i.e. an alcohol comprising one or more denaturants. The alcohol may be ethanol and/or n-propanol. If the indicator material comprises ethanol and/or n-propanol, it may further comprise ethyl acetate. When the indicator comprises a solvent such as ethanol and/or n-propanol, it may comprise a binder such as polyurethane and/or polyamides.

The indicator material may comprise a particulate inorganic substrate, e.g. the dye may be combined with a particulate inorganic substrate. A particulate inorganic substrate is defined herein as a substrate which is typically made of an insoluble material, and which is provided in a particulate form. Examples include inorganic fillers and/or inorganic pigments, which may be white, transparent, or coloured. “Insoluble material” refers to a material that is insoluble in a water-based or organic solvent in which the indicator material is dissolved or suspended, prior to coating and/or impregnating within the particulate inorganic substrate.

The particulate inorganic substrate may be in powder form. Typically, the particulate inorganic substrate may be an inorganic pigment, such as silica, titania, alumina, magnesium oxide, calcium oxide or a zeolite. Preferably, the particulate inorganic substrate may be silica.

In some embodiments, the particulate inorganic substrate may be hydrophobic, such as hydrophobic silica, such as Aerosil® R972 (available from Evonik), or hydrophobic alumina. Hydrophobic particulate inorganic substrates are typically useful as anti-settling agents. Such agents inhibit the indicator material from settling and separating under gravity.

The term “hydrophobic” is understood to mean either inherently hydrophobic, or hydrophobised, i.e. a particulate inorganic substrate which has been modified, e.g. surface-modified, to render it hydrophobic, e.g. by incorporating hydrophobic chemical groups such as alkyl groups on the surface of the particulate inorganic substrate.

In some embodiments, the particulate inorganic substrate may be hydrophilic, e.g. hydrophilic silica, such as Syloid® 244 (available from W.R. Grace & Co), or hydrophilic alumina. Hydrophilic particulate inorganic substrates are typically useful as anti-tack agents. Such agents reduce the cohesion of the indicator material. In other words, anti-tack agents inhibit the indicator material from sticking to itself. This reduces the risk of the indicator material blocking or transferring, making it easier to print the indicator material onto a surface, for example onto the gas barrier layer.

The term “hydrophilic” is understood to mean either inherently hydrophilic, or hydrophilised, i.e. a particulate inorganic substrate which has been modified, e.g. surface-modified, to render the substrate hydrophilic, e.g. by incorporating hydrophilic chemical groups such as hydroxy groups on the surface of the particulate inorganic substrate.

In some embodiments, the particulate inorganic substrate may be an untreated particulate inorganic substrate, such as untreated titania. The particulate inorganic substrate may retain its photocatalytic properties.

The indicator material may comprise a base. This is particularly beneficial when the first substance is carbon dioxide. Suitable bases include any chemical species able to deprotonate the indicator material. Typically, the base may be a hydroxide of formula MOH or M′(OH)₂, wherein M is a monocation and M′ is a dication. Typically, M is any monocation selected from the group consisting of R₄N⁺, K⁺, Na⁺, Cs⁺, Li⁺ and Rb⁺, wherein each R is independently a C₁-C₈alkyl group. Typically, M′ is any dication selected from the group consisting of Ba²⁺, Sr²⁺ and Ca²⁺. Often, the base is of formula MOH. Commonly, M is selected from the group consisting of R₄N⁺, K⁺ and Na⁺, wherein each R is independently a C₁-C₈alkyl group. Typically, each R is independently a C₄-C₈ alkyl group. Often, each R group is the same. The base may be selected from any one of the group consisting of tetrabutylammonium hydroxide, potassium hydroxide, sodium hydroxide and tetraoctylammonium hydroxide. In some embodiments, the base may be tetrabutylammonium hydroxide.

The base may improve the sensitivity of the indicator material to the change in concentration of carbon dioxide. The protons produced when the carbon dioxide dissolves in a protic solvent lower the pH of the environment within the indicator material. Consequently, a higher concentration of carbon dioxide increases the acidity of the environment within the indicator material and vice versa. Indicator materials of the self-supporting film that are sensitive to pH changes may exist in a first chemical state, such as a protonated form of a first colour, at low pH and a second chemical state, such as a deprotonated form of a second colour, at high pH. High concentrations of carbon dioxide are likely to favour the protonated form of a first colour, whereas low concentrations of carbon dioxide are likely to favour the deprotonated form of a second colour. The base may ensure that the indicator material is present in its deprotonated form, which may then be protonated at high concentrations of carbon dioxide. In some embodiments, the indicator material is pH sensitive, typically of a first colour at high concentrations of carbon dioxide and a second colour at low concentrations of carbon dioxide.

The molar ratio of the base to the material sensitive to the concentration of the first substance, such as the reactive dye, may have an effect on the degree of and rate of signal change, such as colour change. For example, the indicator material may comprise a reactive dye that is one colour when deprotonated by the base, and another colour when protonated (e.g. by the protons generated on reaction of carbon dioxide with a protic solvent). Assuming that one molecule of base is able to deprotonate 1 molecule of reactive dye, a ratio of reactive dye to base of >1:1 may lead to the presence of some protonated dye before exposure to protons generated by reaction of a first substance (e.g. carbon dioxide), thus the initial colour of the dye may lie somewhere between the colour of the deprotonated form and the protonated form. This means that, on exposure to protons generated by reaction of a first substance, the change in colour of the dye may be less apparent than that observed when the initial colour of the dye is that of the deprotonated form.

On the other hand, a ratio of reactive dye to base of 1:>>1 is likely to produce deprotonated reactive dye molecules. The initial colour of the dye is likely to be that of the deprotonated form such that the change in colour of the dye on exposure to protons, generated by reaction of a first substance, is apparent. However, a large excess of base with respect to dye may lead to a delay in colour change on exposure of the reactive dye to protons, since the protons may preferentially react with the excess base in the indicator material rather than the deprotonated reactive dye. Consequently, a greater concentration of protons may be required in order to protonate the reactive dye and induce a colour change.

If the indicator material comprises a base and a reactive dye, then the molar ratio of reactive dye to base may be from about 1:1 to about 1:10, about 1:1 to about 1:8, about 1:1 to about 1:6, about 1:1 to about 1:4, or about 1:1 to about 1:3. Typically, the molar ratio of reactive dye to base may be about 1:2.

The indicator material may comprise or may be combined with a resin. The resin may be any resin suitable to seal and protect the indicator material. If the first substance is carbon dioxide and the indicator material comprises a base, then it is preferable that the resin has an acid value of no more than 20, more preferably no more than 15, and even more preferably no more than 10. The term “acid value” is used herein to refer to the mass of potassium hydroxide (in mg) that is required to neutralise one gram of the material (in this case, the resin). By “neutralise” is meant that each acidic group in the material has reacted with the potassium hydroxide such that there is no excess of hydrogen ions or hydroxide ions in the resultant composition. The resin may be any one selected from the group consisting of a self-crosslinking acrylic emulsion, such as Joncryl® FLX 5010 (available from BASF), a self-crosslinking styrene-acrylic emulsion, a self-crosslinking styrene emulsion, an acrylic-styrene emulsion, an acrylic emulsion, a styrene emulsion and a polyurethane emulsion. Typically, the resin is any one selected from the group consisting of a self-crosslinking acrylic emulsion, such as Joncryl® FLX 5010, a self-crosslinking styrene-acrylic emulsion and a polyurethane emulsion. In an embodiment, the resin may be a self-crosslinking acrylic emulsion, such as Joncryl® FLX 5010.

The indicator material may be as described in GB 2 474 571 (Mills et an, the contents of which are incorporated herein by reference in their entirety.

The indicator material of the self-supporting film may be in direct contact with the gas barrier layer or the semi-permeable layer, i.e. there may not be an adhesive layer or any other layer positioned between the indicator material and the gas barrier layer or the semi-permeable layer. The indicator material, and any materials combined with it, may be applied, e.g. printed, directly onto the gas barrier layer or the semi-permeable layer. Alternatively, the indicator material may be laminated between the gas barrier layer and the semi-permeable layer. Lamination may be conducted at high temperatures, thus requiring the indicator material to have a high thermal stability, such as at least approximately 80° C., preferably at least 110° C. Preferably, the indicator material may be printed directly onto the gas barrier layer or the semi-permeable layer. The printing may be carried out using a wide web flexographic printing press, advantageously at ambient temperature, and consequently may not require the chemical indicator to have a high thermal stability.

Preferably, and in accordance with the second and fourth aspects, the indicator material may be in direct contact with the gas barrier layer. Typically, the indicator material may be applied, e.g. printed, directly onto the gas barrier layer.

The self-supporting film may not comprise a release layer or an outer adhesive layer. By “release layer” is meant a layer (typically an outer layer) suitable for detachment from the self-supporting film. By such provision, the self-supporting film may not require it to be applied to a separate support or substrate, as for example in the case of labels or stickers.

The self-supporting film may comprise at least one reference material. By “reference material” is meant a material that displays a signal, such as a colour, for example the signal or colour of the indicator material at a specific concentration of a first substance or after exposure to the first substance for a predetermined amount of time. Thus, the reference material may be compared with the indicator material to determine the concentration of the first substance present at the indicator material or the amount of time after which the indicator has been exposed to the first substance.

Typically, the at least one reference material is coloured, wherein the colour corresponds to the colour of the indicator material at a specific concentration of first substance or after exposure to the first substance for a predetermined amount of time. The at least one reference material is typically provided near or around the indicator material, e.g. in the same layer as the indicator material, by which is meant that the at least one reference material is located at the same depth of the self-supporting film as the indicator material. Typically, the at least one reference material may be positioned at the same depth as the indicator material, and adjacent or near to the indicator material. Accordingly, the at least one reference material may typically be positioned between the gas barrier layer and the semi-permeable layer. Typically, the reference material may be positioned in discrete sections of the self-supporting film. As with the indicator material, the at least one reference material may be in direct contact (by printing or laminating methods, typically printing) with the gas barrier layer or the semi-permeable layer. Preferably, the at least one reference material may be applied, e.g. printed, directly onto the gas barrier layer.

In some embodiments, the at least one reference material may have the same composition as the indicator material before exposure to the first substance or a certain time after exposure to the first substance. In these embodiments, the reference material may be sealed from the first substance to avoid any changes in composition.

Typically, the reference material does not comprise an/the indicator material. That is, the colour of the reference material may not be altered by a change in concentration of the first substance.

The self-supporting film may comprises plurality of reference materials, e.g. three reference materials.

The reference materials may be applied to the self-supporting film in such a way to expose a contrast material positioned below the reference material and reveal a label or text. Alternatively, the reference materials may be printed together with an inert ink of a different colour in order to label the reference material, for example with text.

The self-supporting film may comprise an adhesive layer. The adhesive layer may typically be provided adjacent to the semi-permeable layer, e.g. between the indicator material and the semi-permeable layer. The adhesive layer may be permeable to a/the first substance.

Thus, in some embodiments, the self-supporting film may comprise (in order of depth):

a gas barrier layer;

an indicator material;

an adhesive layer; and

a semi-permeable layer.

One or more reference materials may be positioned at the substantially same depth as the indicator material, thus the adhesive layer may be adjacent to both the indicator material and the one or more reference materials.

The adhesive layer may comprise any adhesive suitable to contact, e.g. permanently or irreversibly contact, the semi-permeable layer with the indicator material and optional reference materials. If the first substance is carbon dioxide and the indicator material comprises a base, then it is preferable that the adhesive has a suitably low acid value to prevent interference with the indicator material. The adhesive may be a polyurethane adhesive, typically a two-part polyurethane adhesive such as CA3278/7+SF3277/3 or SF707A+CA336. In some embodiments, the adhesive may be CA3278/7+SF3277/3. It will be understood that any other adhesive may be used, provided it is compatible with the layers to which it is bonded, e.g. the indicator material.

Adhesives may release carbon dioxide on curing. If the first substance is carbon dioxide then it may be useful to avoid adhesives that, on curing, release high quantities of carbon dioxide, as these may interfere with the indicator material. Often, highly humid environments are avoided when applying the adhesive layer to the self-supporting film, since a greater humidity may promote carbon dioxide production on curing the adhesive.

To avoid exposure of the indicator material to carbon dioxide that may be released from the adhesive layer on curing, the adhesive layer may not be in contact with the indicator material. Rather, the adhesive layer may only contact the reference material, the semi-permeable layer and/or the gas barrier layer. Accordingly, the adhesive layer may be non-continuous/patterned. Advantageously, the adhesive layer may be absent in the sections or areas of the self-supporting film that comprise the indicator material. In other words, the adhesive layer may be adjacent to the semi-permeable layer and/or may bond the semi-permeable layer ad the gas barrier layer, in the sections of the self-supporting film that do not comprise the indicator material. Alternatively, the adhesive layer may be continuous.

Typically, the adhesive layer may span substantially the entire surface area of the self-supporting film.

The self-supporting film may further comprise a contrast material to improve the visibility of the indicator material and/or of at least one reference material (if present). The contrast material may be permeable to the first substance.

The contrast material may be combined with the indicator material and/or at least one reference material (if present). Alternatively, the contrast material may be positioned adjacent to the indicator material and/or at least one reference material (if present), typically on a side of the indicator material opposite the gas barrier layer. The contrast material may be positioned between the indicator material and/or, if present, at least one reference material and an adhesive layer.

If the first substance is carbon dioxide and the indicator material comprises a base, then it is preferable that the contrast material has a suitably low acid value to prevent interference with the indicator material. In some embodiments, the contrast material of the self-supporting film may be opaque. The contrast material may comprise a pale-coloured ink. Typically, the contrast layer may comprise a white pigment, such as titanium dioxide.

The gas barrier layer of the self-supporting film may comprise a coating.

Advantageously, the coating may enhance the properties of the gas barrier layer. Beneficially, the coating may reduce the permeability of the gas barrier layer to a first substance and/or oxygen.

Thus, the gas barrier layer may comprise or made be made of two or more layers, e.g. two layers. The gas barrier layer may comprise or made be made of at least one base barrier layer and at least one coating layer. The gas barrier layer may comprise or made be made of a barrier layer and a coating layer. Typically, the at least one coating layer may have a gas permeability less than the gas permeability of the base barrier layer, for example in relation to the first substance. By such provision, the base barrier layer(s) may comprise, may consist essentially of, or may consist of a material with a relatively high permeability, whilst the at least one coating layer may provide the gas barrier layer with an overall sufficiently low gas permeability, e.g. for example a sufficiently low carbon dioxide transmission rate, oxygen transmission rate and/or water vapour transmission rate.

The coating may have a carbon dioxide transmission rate of less than about 10 cc/m²/day, e.g. less than about 8 cc/m²/day, e.g. less than about 5 cc/m²/day. The coating may have a carbon dioxide transmission rate of about 0.1 to about 10 cc/m²/day, about 0.1 to about 8 cc/m²/day or about 0.1 to about 5 cc/m²/day.

The coating may have an oxygen transmission rate of less than about 10 cc/m²/day, e.g. less than about 5 cc/m²/day, e.g. less than about 1 cc/m²/day. The coating may have an oxygen transmission rate of about 0.05 to about 10 cc/m²/day, about 0.05 to about 5 cc/m²/day or about 0.1 to about 2 cc/m²/day.

The coating may have a water vapour transmission rate of less than about 30 g/m²/day, e.g. less than about 20 g/m²/day, e.g. less than about 10 g/m²/day, e.g. less than about 5 g/m²/day. The coating may have a water vapour transmission rate from about 0.01 to about 30 g/m²/day, about 0.01 to about 20 g/m²/day, about 0.05 to about 10 g/m²/day, or about 0.1 to about 5 cc/m²/day.

The coating may have a carbon dioxide transmission rate of about 0.1 to about 5 cc/m²/day, an oxygen transmission rate of about 0.1 to about 2 cc/m²/day and a water vapour transmission rate of about 0.1 to about 5 cc/m²/day.

The coating may be positioned on the outer surface of the gas barrier layer, or on the inner surface of the gas barrier layer. Typically, the coating may be positioned on the inner surface of the gas barrier layer, and/or adjacent to the indicator material. The coating may be or may comprise any material suitable to reduce the permeability of the gas barrier layer to a first substance and/or oxygen. Typically, the coating may be or may comprise any one selected from the group consisting of aluminium oxide, polyvinylidene dichloride and ethylene vinyl alcohol. In an embodiment, the coating may be aluminium oxide.

If the first substance is carbon dioxide, the indicator material comprises a base, and the coating is positioned adjacent to the indicator material, then it is preferable that the coating has a suitably low acid value to prevent interference with the indicator material.

The self-supporting film may further comprise an outer sealing layer positioned adjacent to the semi-permeable layer. The outer sealing layer may span substantially the entire surface area of the self-supporting film. The outer sealing layer may be permeable to a first substance. The purpose of the sealing layer may be to seal the self-supporting film to a container, such as a tray. Typically, the sealing layer comprises the same material composition as the container. Typically, the sealing layer may comprise polypropylene.

The sealing layer may comprise a heat-seal coating, which may be permeable to a first substance. The heat-seal coating may typically be positioned on the outer surface of the sealing layer. The heat-seal coating may be suitable to seal the self-supporting film to a container, such as a tray, when heat is applied.

Typically, the self-supporting film may comprise an upper outer gas barrier layer, a lower outer semi-permeable layer, an indicator material provided on an inner side of the gas barrier layer, and an adhesive layer provided between the indicator material and the inner semi-permeable layer. In the third aspect, the gas barrier layer and semi-permeable layer are of substantially equal surface area, whilst in the fourth aspect, the indicator material is in direct contact with the gas barrier layer.

The self-supporting film may be a packaging film. By “packaging film” is meant any film suitable for use in wrapping or protecting goods.

In accordance with the fifth aspect, there is provided an item of packaging comprising the self-supporting film of ay one of the first to fourth aspects, and in accordance with the sixth aspect, there is provided use of the self-supporting film according to any one of the first to fourth aspects, in an item of packaging, optionally food packaging. The item of packaging may be any item suitable for wrapping or protecting goods, such as a sealed container, box, bag or wrap. Typically, the item of packaging is Modified Atmosphere Packaging (‘MAP’), flushed with carbon dioxide. Typically the MAP comprises at least about 10% carbon dioxide, often at least about 20% carbon dioxide, and most often at least about 30% carbon dioxide. The MAP may comprise up to 100% carbon dioxide, for example at least about 40%, at least about 50%, at least about 60%, at least about 70%, at least about 80% or at least about 90% carbon dioxide.

Typically, the self-supporting film is a food packaging film. The food packaging film may be suitable for packaging perishable foods, i.e. the packaging film is a perishable food packaging film. The perishable food may be any food with a use by date that falls within one month of opening the packaging. Typically, the food is any food with a use by date that falls within two weeks, more typically 1 week, of opening the packaging.

The perishable food may be any one selected from the group consisting of cooked meats, raw meats, cheese and fresh produce. “Fresh produce” is used herein to refer to fresh farm-produced crops, such as fresh fruit and/or fresh vegetables.

The self-supporting film may be useful in determining the amount of time that has passed since the exposure of a perishable food to a first material, such as oxygen, or since the reduced exposure of a perishable food to a first substance, such as carbon dioxide. Consequently, the self-supporting film may be useful in identifying faults in the packaging of perishable food, or determining the amount of time that has passed since the packaging of perishable food has been opened.

According to the seventh aspect, there is provided a method of manufacturing a self-supporting film, the method comprising:
(i) providing a gas barrier layer;
(ii) providing a semi-permeable layer;
(iii) applying an indicator material onto the gas barrier layer or the semi-permeable layer; and
(iv) bonding and/or laminating the gas barrier layer and the semi-permeable layer;

wherein the indicator material is positioned between the gas barrier layer and the semi-permeable layer.

The method may comprise printing the indicator material onto the gas barrier layer or the semi-permeable layer. Typically, the method may comprise printing the indicator material onto the gas barrier layer.

The method may comprise bonding the gas barrier layer on which the indicator material is applied, and the semi-permeable layer.

The method may comprise applying an adhesive onto at least one of the gas barrier layer and the semi-permeable layer. If the indicator material is printed directly onto the gas barrier layer, then the adhesive may be applied onto the semi-permeable layer and vice versa.

The method may comprise applying the/an adhesive onto the gas barrier layer, e.g. onto the gas barrier layer provided with the indicator material. The method may comprise applying the adhesive onto the inner or lower side and/or onto the indicator side.

The terms “lower” and “inner” are not to be construed in an absolute sense, but refer to the side of the film or packaging film, in use, relative to a respective container or contents thereof.

The method may comprise applying the/an adhesive onto the semi-permeable layer, e.g. onto an upper or outer side thereof.

The terms “upper” and “outer” are not to be construed in an absolute sense, but refer to the side of the film or packaging film, in use, relative to a respective container or contents thereof.

The method may comprise applying the/an adhesive substantially over the entire surface of the gas barrier layer provided with the indicator material and/or the semi-permeable layer.

The method may comprise applying the/an adhesive substantially over a discrete surface of the gas barrier layer provided with the indicator material and/or the semi-permeable layer, e.g. over a surface thereof not aligned with the indicator material. By such provision, the adhesive may not interfere with the indicator material, for example when the adhesive may be susceptible to releasing chemicals, e.g. gases, upon curing.

The method may comprise bringing the gas barrier layer and the semi-permeable layer together, e.g. bringing an outer or upper side of the semi-permeable layer into contact with a lower or inner side of the gas barrier layer provided with the indicator, at least one of which being provided with the/an adhesive.

The method may comprise laminating, e.g. heat laminating, the gas barrier layer provided with the indicator material, and the semi-permeable layer.

The method may comprise applying pressure. The method may comprise passing the film through a calender and/or two or more rollers.

The features described in relation to any one aspect may apply in relation to any other aspect, and are not repeated in each aspect merely for brevity.

Each and every patent and non-patent reference referred to herein is hereby incorporated by reference in its entirety, as if the entire contents of each reference were set forth herein in their entirety.

The invention may be further described with reference to the following non-limiting clauses:

1. A self-supporting film comprising:
(i) a gas barrier layer;
(ii) a semi-permeable layer; and
(iii) an indicator material positioned between the gas barrier layer and the semi-permeable layer;
wherein the gas barrier layer and semi-permeable layer are of substantially equal surface area.
2. The self-supporting film of clause 1, wherein the indicator material is in direct contact with the gas barrier layer.
3. A self-supporting film comprising:
(i) a gas barrier layer;
(ii) a semi-permeable layer; and
(iii) an indicator material provided between the gas barrier layer and the semi-permeable layer;
wherein the indicator material is in direct contact with the gas barrier layer.
4. The self-supporting film of clause 3, wherein the gas barrier layer and semi-permeable layer are of substantially equal surface area.
5. The self-supporting film of any one preceding clause, wherein the self-supporting film does not comprise a release layer or an outer adhesive layer.
6. The self-supporting film of any one preceding clause, wherein the indicator material is a colourimetric indicator material and/or wherein the indicator material is capable of changing colour when exposed to a first substance.
7. The self-supporting film of any one preceding clause, wherein the indicator material is sensitive to the concentration of a/the first substance.
8. The self-supporting film of any one preceding clause, wherein the gas barrier layer has a permeability that is low enough to provide an acceptable barrier to a/the first substance.
9. The self-supporting film of any one preceding clause, wherein the semi-permeable layer has a permeability that allows for a controlled flow of a/the first substance.
10. The self-supporting film of any one preceding clause, wherein the gas barrier layer is less permeable to a/the first substance than the semi-permeable layer.
11. The self-supporting film of any one of clauses 1 to 9, wherein the gas barrier layer is at least 50%, 60%, 70% or 80% less permeable to a first substance than the semi-permeable layer.
12. The self-supporting film of any one of clauses 6 to 11, wherein the first substance is carbon dioxide, oxygen, water or ammonia.
13. The self-supporting film of any one of clauses 6 to 11, wherein the first substance is carbon dioxide.
14. The self-supporting film of any one preceding clause, wherein the gas barrier layer has an oxygen transmission rate of ≤about 40 cc/m²/day, ≤about 20 cc/m²/day, ≤about 10 cc/m²/day, or ≤about 5 cc/m²/day.
15. The self-supporting film of any one of clauses 1 to 13, wherein the gas barrier layer has an oxygen transmission rate from about 0.01 to about 40 cc/m²/day, about 0.05 to about 20 cc/m²/day, about 0.1 to about 10 cc/m²/day, or about 0.1 to about 5 cc/m²/day.
16. The self-supporting film of any one of clauses 1 to 13, wherein the gas barrier layer has an oxygen transmission rate of about 2 cc/m²/day.
17. The self-supporting film of any one preceding clause, wherein the semi-permeable layer has an oxygen transmission rate of ≤about 4000 cc/m²/day, ≤about 1000 cc/m²/day, ≤about 150 cc/m²/day, ≤about 125 cc/m²/day, ≤about 100 cc/m²/day, or ≤about 80 cc/m²/day.
18. The self-supporting film of any one of clauses 1 to 16, wherein the semi-permeable layer has an oxygen transmission rate from about 30 to about 200 cc/m²/day, about 35 to about 160 cc/m²/day, about 40 to about 150 cc/m²/day, or about 45 to about 80 cc/m²/day.
19. The self-supporting film of any one of clauses 1 to 18, wherein the semi-permeable layer has an oxygen transmission rate of about 60 cc/m²/day.
20. The self-supporting film of any one preceding clause, wherein the gas barrier layer has a carbon dioxide transmission rate of ≤about 20 cc/m²/day, ≤about 15 cc/m²/day, ≤about 10 cc/m²/day, or ≤about 5 cc/m²/day.
21. The self-supporting film of any one of clauses 1 to 19, wherein the gas barrier layer has a carbon dioxide transmission rate from about 0.01 to about 20 cc/m²/day, about 0.05 to about 15 cc/m²/day, about 0.1 to about 10 cc/m²/day, or about 0.15 to about 5 cc/m²/day.
22. The self-supporting film of any one of clauses 1 to 19, wherein the gas barrier layer has a carbon dioxide transmission rate of about 3 cc/m²/day.
23. The self-supporting film of any one preceding clause, wherein the semi-permeable layer has a carbon dioxide transmission rate of ≤about 4000 cc/m²/day, ≤about 1000 cc/m²/day, ≤about 150 cc/m²/day, ≤about 125 cc/m²/day, ≤about 100 cc/m²/day, ≤about 80 cc/m²/day, or ≤about 40 cc/m²/day.
24. The self-supporting film of any one of clauses 1 to 22, wherein the semi-permeable layer has a carbon dioxide transmission rate from about 5 to about 200 cc/m²/day, about 5 to about 160 cc/m²/day, about 10 to about 150 cc/m²/day, or about 10 to about 40 cc/m²/day.
25. The self-supporting film of any one of clauses 1 to 22, wherein the semi-permeable layer has a carbon dioxide transmission rate of about 20 cc/m²/day.
26. The self-supporting film of any one preceding clause, wherein the indicator material comprises any one of the group consisting of m-Cresol Purple, thymolphthalein, o-Cresolphthalein, acryloly florescein, β-methyl umbelliferon, Bromothymol blue, 5′ and 6-Carboxyseminaphtholfluorescein, 5′ and 6′-Carboxyseminaphtholrhodamine, Cresol Red, 2-(2,4-Dinitrophenylazo)-1-naphthol-3,6-disulphonic acid, tris(thenoyltrifluoroacetonato) europium (III), Fluorescein, 7-hydroxycoumarin-4-acetic acid, 8-hydroxypyrene-1,3,6-trisulphonic acid, Neutral red, Phenol Red, Rhodamine 6G, Sulforhodamine 101, Thymol blue, and Texas Red hydrazine.
27. The self-supporting film of any one of clauses 1 to 26, wherein the indicator material is selected from any one of the group selected from Cresol Red, m-Cresol Purple, Phenol Red and Thymol blue.
28. The self-supporting film of any one preceding clause, wherein the indicator material comprises a base, such as tetrabutylammonium hydroxide.
29. The self-supporting film of any one preceding clause, wherein the indicator material comprises a particulate inorganic substrate, such as hydrophobic silica or alumina, and/or hydrophilic silica or alumina.
30. The self-supporting film of any one preceding clause, wherein the indicator material comprises or is combined with a resin, such as a self-crosslinking acrylic emulsion.
31. The self-supporting film of any one preceding clause, wherein the self-supporting film further comprises at least one reference material.
32. The self-supporting film of clause 31, wherein the at least one reference material is coloured.
33. The self-supporting film of clause 31 or 32, wherein the at least one reference material is provided near or around the indicator material.
34. The self-supporting film of any one of clauses 31 to 33, wherein the at least one reference material is provided in the same layer as the indicator material.
35. The self-supporting film of clause 34, wherein the at least one reference material is positioned at the same depth as the indicator material, and adjacent to the indicator material.
36. The self-supporting film of any one of clauses 31 to 35, wherein the reference material is positioned between the gas barrier layer and the semi-permeable layer.
37. The self-supporting film of any one preceding clause further comprising an adhesive layer.
38. The self-supporting film of clause 37, wherein the adhesive layer is adjacent to the semi-permeable layer and/or is between the semi-permeable layer and the indicator material.
39. The self-supporting film of clause 37 or clause 38, wherein the indicator material is positioned between the gas barrier layer and the adhesive layer.
40. The self-supporting film of any one of clauses 37 to 39, wherein the adhesive layer is non continuous and/or is provided in a pattern.
41. The self-supporting film of any one of clauses 37 to 40, wherein the adhesive layer is not in contact with the indicator material.
42. The self-supporting film of any one of clauses 37 to 41, further comprising a contrast material positioned between the indicator material and the adhesive layer.
43. The self-supporting film of any one of clauses 1 to 41, further comprising a contrast material adjacent to the indicator material, optionally on a side of the indicator material opposite the gas barrier layer.
44. The self-supporting film of clause 42 or clause 43, wherein the contrast material improves the visibility of the indicator material.
45. The self-supporting film of any one of clauses 42 to 44, wherein the contrast material is opaque.
46. The self-supporting film of any one of clauses 42 to 45, wherein the contrast material comprises a pale-coloured ink.
47. The self-supporting film of any one of clauses 42 to 45, wherein the contrast material comprises a white pigment, such as titanium dioxide.
48. The self-supporting film of any one preceding clause, wherein the gas barrier layer comprises a coating.
49. The self-supporting film of clause 48, wherein the coating reduces the permeability of the gas barrier layer to a first substance and/or oxygen.
50. The self-supporting film of clause 48 or clause 49, wherein the coating is any one selected from the group consisting of aluminium oxide, polyvinylidene dichloride and ethylene vinyl alcohol.
51. The self-supporting film of any one preceding clause, wherein the gas barrier layer comprises polyethylene terephthalate.
52. The self-supporting film of any one preceding clause, wherein the semi-permeable layer comprises polyethylene terephthalate.
53. The self-supporting film of any one preceding clause, further comprising a sealing layer adjacent to the semi-permeable layer.
54. The self-supporting film of clause 53, wherein the sealing layer comprises a heat-seal coating.
55. The self-supporting film of clause 53 or clause 54, wherein the sealing layer is suitable to seal the film to a tray.
56. The self-supporting film of any one preceding clause, wherein the gas barrier layer is transparent or semi-transparent.
57. A self-supporting film comprising:
(i) a upper outer gas barrier layer;
(ii) a lower outer semi-permeable layer;
(iii) an indicator material provided on an inner side of the gas barrier layer; and
(iv) an adhesive layer provided between the indicator material and the inner semi-permeable layer;
wherein the gas barrier layer and semi-permeable layer are of substantially equal surface area.
58. A self-supporting film comprising:
(i) a upper outer gas barrier layer;
(ii) a lower outer semi-permeable layer;
(iii) an indicator material provided on an inner side of the gas barrier layer; and
(iv) an adhesive layer provided between the indicator material and the inner semi-permeable layer;
wherein the indicator material is in direct contact with the gas barrier layer.
59. The self-supporting film according to any one preceding clause, where the self-supporting film is a packaging film.
60. An item of packaging comprising the packaging film of clause 59.
61. The self-supporting film or item of packaging according to clause 59 or clause 60, wherein the packaging film is a food packaging film.
62. Use of the self-supporting film according to any one of clauses 1 to 59 and 61, in an item of packaging, optionally food packaging.
63. A method of manufacturing a self-supporting film, the method comprising:
(i) providing a gas barrier layer;
(ii) providing a semi-permeable layer;
(iii) applying an indicator material onto the gas barrier layer or the semi-permeable layer; and
(iv) bonding and/or laminating the gas barrier layer and the semi-permeable layer;

wherein the indicator material is positioned between the gas barrier layer and the semi-permeable layer.

### BRIEF DESCRIPTION OF DRAWINGS

Embodiments of the invention will now be given by way of example only, and with reference to the accompanying drawings, in which:

FIG. 1 is a cross-section of a self-supporting film according to a first embodiment;

FIG. 2 is a cross-section of a self-supporting film according to a second embodiment, wherein the gas barrier layer comprises a coating;

FIG. 3 is a cross-section of a self-supporting film according to a third embodiment, including reference materials;

FIG. 4 is a cross-section of a self-supporting film according to a fourth embodiment, including an adhesive layer positioned between the indicator material and the semi-permeable layer;

FIG. 5 is a cross-section of a self-supporting film according to a fifth embodiment, including an adhesive layer positioned between the indicator material and the semi-permeable layer;

FIG. 6 is a cross-section of a self-supporting film according to a sixth embodiment, including a contrast material and an adhesive layer that are adjacent to one another;

FIG. 7 is a cross-section of a self-supporting film according to a seventh embodiment, including a sealing layer, which allows the self-supporting film to seal to a container;

FIG. 8 is a working example of a self-supporting film according to another embodiment;

FIG. 9 is a graph of the hue on the yellow/blue axis of the indicator material as a function of time (days) for a working example of a self-supporting film.

### DETAILED DESCRIPTION

The following are examples of specific embodiments.

### Drawings

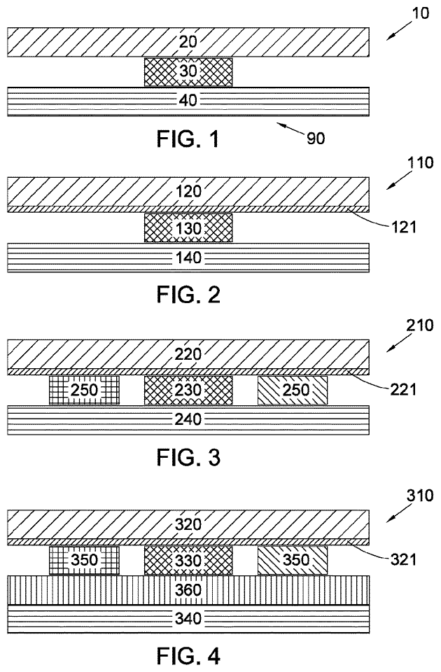

FIG. 1 is a schematic cross-section of a self-supporting film 10 according to a first embodiment, comprising a gas barrier layer 20, a semi-permeable layer 40, and an indicator material 30 positioned between gas barrier layer 20 and semi-permeable layer 40. Gas barrier layer 20 and semi-permeable layer 40 are of substantially equal surface area and indicator material 30 is in direct contact with gas barrier layer 20.

Indicator material 30 is positioned in a discrete section of film 10, between gas barrier layer 20 and semi-permeable layer 40. It is to be understood that indicator material 30 of film 10 may span substantially the entire surface area of film 10, by which is meant that indicator material 30 may extend across at least 95% of the surface area of film 10. Typically, however, indicator material 30 spans a smaller surface area than gas barrier layer 20 and semi-permeable layer 40, and is positioned in discrete sections of film 10.

A first substance in an environment 90, positioned below semi-permeable layer 40 is able to flow to or from indicator material 30, but is blocked by gas barrier layer 20. If environment 90 changes, by increasing or decreasing the concentration of the first substance, then indicator material 30 may sense this change in a time-controlled manner, at a rate that is dependent on the flow of the first substance through semi-permeable layer 40.

FIG. 2 is a cross-section of a self-supporting film 110 according to a second embodiment. Film 110 is generally similar to film 10, like part denoted by like numerals, but incremented by ‘100’, and comprises a gas barrier layer 120, a semi-permeable layer 140, and an indicator material 130 positioned between gas barrier layer 120 and semi-permeable layer 140. Gas barrier layer 120 and semi-permeable layer 140 are of substantially equal surface area and indicator material 130 is in direct contact with gas barrier layer 120. However, in this embodiment, gas barrier layer 120 comprises a coating 121. Coating 121 preferably spans substantially the entire surface area of the self-supporting film. It is to be understood that coating 121 may be positioned on the outer surface of gas barrier layer 120, or on the inner surface of gas barrier layer 120. However, coating 121 is typically positioned on the inner surface of gas barrier layer 120, adjacent to indicator material 130, as shown in FIG. 2. Coating 121 may decrease the permeability of gas barrier layer 120 to a first substance in order to prevent gas flow through gas barrier layer 120.

FIG. 3 is a schematic cross-section of a self-supporting film 210 according to a third embodiment. Film 210 is similar to film 110, like part denoted by like numerals, but incremented by ‘100’, and comprises a gas barrier layer 220, a semi-permeable layer 240, an indicator material 230 positioned between gas barrier layer 220 and semi-permeable layer 240, and a coating 221. In this embodiment, the film 210 further comprises reference materials 250 positioned in discrete sections of film 210, in the same layer as indicator material 230, and adjacent to indicator material 230. Any number of reference materials 250 may be positioned adjacent to indicator material 230. Conveniently, reference materials 250 may assist the user in assessing the concentration of a first substance at indicator material 230. However, when not present, a user could instead refer to a separate reference scale, or simply estimate the concentration.

The change in signal from indicator material 230 is time-controlled. Reference materials 250 may be used to assess the concentration of the first substance at indicator material 230, which corresponds to the amount of time that has passed since the concentration of the first substance changed.

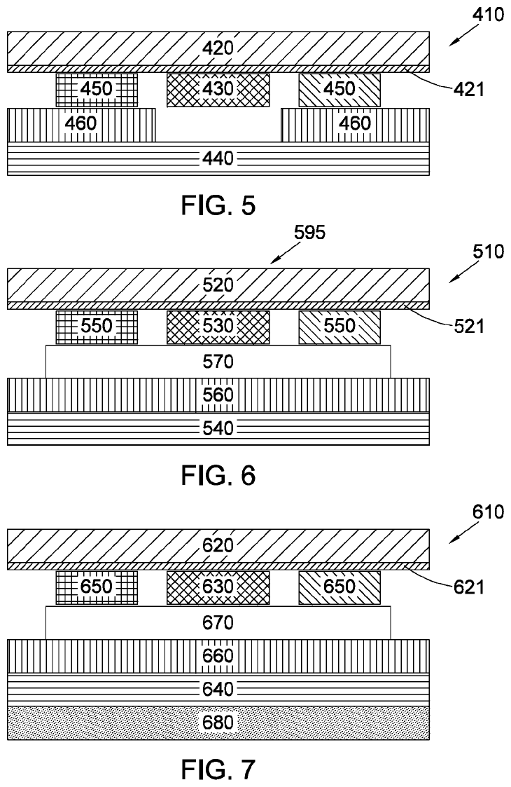

FIG. 4 and FIG. 5 each show a schematic cross-section of a self-supporting film 310 and 410, according to a fourth a fifth embodiment respectively. Films 310 and 410 are similar to film 210, like part denoted by like numerals, but incremented by ‘100’ ad ‘200’ respectively, and each comprise a gas barrier layer 320,420, a semi-permeable layer 340,440, an indicator material 330,430 positioned between gas barrier layer 320,420 and semi-permeable layer 340,440, a coating 321,421 and reference materials 350,450, positioned in discrete sections of each film 310,410 in the same layer as indicator material 330/430.

Films 310 and 410 further comprise an adhesive layer 360,460 positioned between indicator material 330,430 and respective semi-permeable layer 340,440. It is to be understood that adhesive layer 360,460 could alternatively be positioned between coating 321,421 and indicator material 330,430. In the embodiment of FIG. 4, the adhesive layer 360 is continuous, i.e. it spans substantially the entire surface area of film 310. In the embodiment of FIG. 5, the adhesive layer 460 is non-continuous/patterned. Some adhesives are known to release carbon dioxide on curing. A benefit to adhesive layer 460 over adhesive layer 360 is that it may be positioned within film 410 so that it is not in contact with indicator material 430. This avoids any interference to indicator material 430 that may result from carbon dioxide release when the adhesive layer cures. Owing to ease of manufacture, a continuous adhesive layer such as 360 is more convenient and/or less expensive.

FIG. 6 is a schematic cross-section of a self-supporting film 510 according to a sixth embodiment. Film 510 is similar to film 310, like part denoted by like numerals, but incremented by ‘200’ and comprises a gas barrier layer 520, a semi-permeable layer 540, an indicator material 530 positioned between gas barrier layer 520 and semi-permeable layer 540, a coating 521, reference materials 550, positioned in discrete sections of film 510 in the same layer as indicator material 530, and an adhesive layer 560.

In this embodiment, the film 510 further comprises a contrast material 570 positioned between indicator material 530 and adhesive layer 560. Adhesive layer 560 is positioned between contrast layer 570 and semi-permeable layer 540. It is to be understood that adhesive layer 560 could alternatively be positioned between coating 521 and indicator material 530. Contrast material 570 may enhance the visibility of indicator material 530 to the user. The user typically views film 510 from above the gas-barrier layer, with a user's view 595. To enhance the visibility of indicator 530 to the user, contrast material 570 is positioned at a greater depth than indicator material 530 (otherwise indicator material 530 may be blocked from user's view 595). Alternatively, contrast material 570 may be combined with indicator material 530. Contrast material 570 may also be combined with reference materials 550, although this may not be necessary if reference materials 550 are opaque.

FIG. 7 is a schematic cross-section of a self-supporting film 610 according to a seventh embodiment. Film 610 is similar to film 510, like part denoted by like numerals, but incremented by ‘100’, and comprises a gas barrier layer 620, a semi-permeable layer 640, an indicator material 630 positioned between gas barrier layer 620 and semi-permeable layer 640, a coating 621, reference materials 650, positioned in discrete sections of film 610 in the same layer as indicator material 630, an adhesive layer 660, and a contrast material 670. It is to be understood that adhesive layer 660 could alternatively be positioned between coating 621 and indicator material 630.

In this embodiment, the film 610 further comprises a sealing layer 680, which may allow film 610 to seal to a container, for example by applying heat.

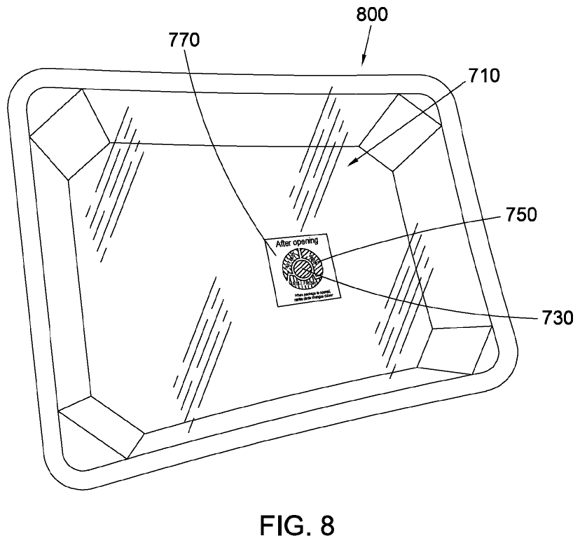

FIG. 8 is an above-view of a working example of a self-supporting film 710 according to an eighth embodiment. The Film 710 is used to seal a container, thus providing a sealed container 800. In this embodiment, an indicator material 730, which comprises Cresol red, is positioned in a circular portion of the film (yellow circle) and three reference materials 750 are positioned in discrete sections of the film in the same layer as and around indicator material 730 (blue, yellow and green arrows). A contrast material 770 (white square) is positioned beneath indicator material 730 and reference materials 750. Film 710 is part of a MAP and the first substance (flushing gas) is carbon dioxide.

Film 710 encloses and seals a 30% carbon dioxide environment, such that the carbon dioxide environment is adjacent to the semi-permeable layer of film 710 and is within the sealed container 800. A standard atmospheric environment is adjacent to the gas barrier layer of film 710 and around sealed container 800. The carbon dioxide environment is able to permeate the semi-permeable layer, the adhesive and contrast material 770, and flows to and from Cresol red indicator material 730. When exposed to a carbon dioxide environment, Cresol red is yellow in colour, and when exposed to standard atmospheric conditions, it is blue in colour. In this embodiment, since Cresol red indicator material 730 is yellow, it may be inferred that the environment around cresol red indicator material 730 is high in carbon dioxide, and therefore that the integrity of the sealed container has not been compromised.

Reference materials 750 are either printed in such a way to expose contrast material 770 and form a label or text (blue and green arrows reading “past best” and “still fresh”, respectively), or are printed together with inert ink of a different colour to form a label or text (yellow arrow reading “fresh” in black ink).

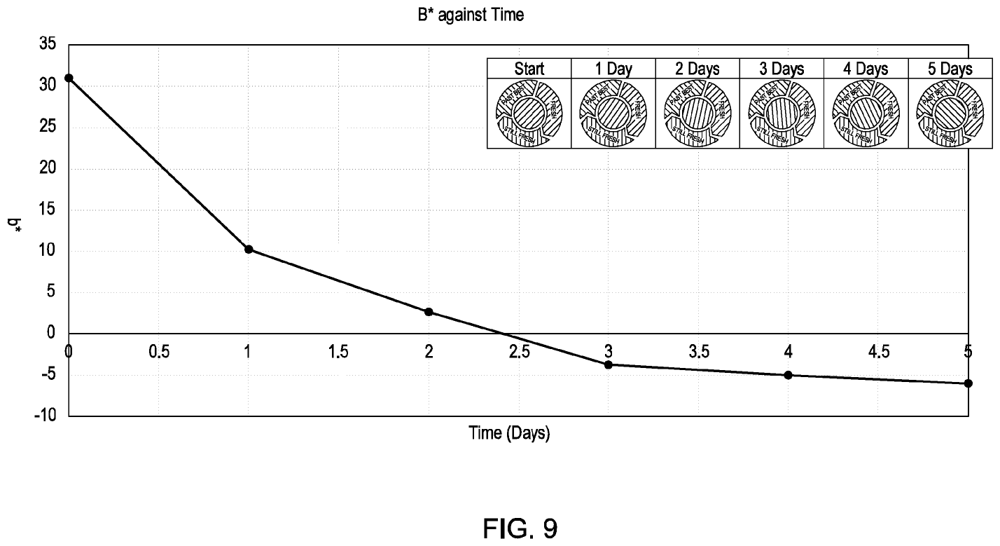

FIG. 9 is a graph showing the hue on the yellow/blue axis (in an L*a*b colour scale) of indicator material 730 as a function of time (days) for the embodiment shown in FIG. 8. The time is measured from opening film 710, i.e. breaking the seal, thereby changing the environment within sealed container 800 from a carbon dioxide environment to a standard atmospheric environment. The environment within container 800 quickly changes—carbon dioxide may diffuse out of the break in the seal and other gases may diffuse into the break, forming a standard atmospheric environment. However, the concentration of carbon dioxide at indicator material 730 changes more slowly, owing to the controlled rate of carbon dioxide flow through the semi-permeable layer.

FIG. 9 shows that the b* value (the hue on the yellow/blue axis) decreases with time. In accordance with the conventional L*a*b colour scale, a more positive b* value corresponds to a more yellow colour, whereas a more negative b* value corresponds to a more blue colour. Thus, the decrease in the b* value corresponds to cresol red indicator material 730 becoming more blue in colour, as shown in the photographs of the insert. This is in agreement with a decreasing concentration of carbon dioxide at cresol red indicator material 730. In this example, the colour change of cresol red indicator material 730 takes approximately 3 days. Consequently, self-supporting film 710 may be useful as an indicator of the freshness of perishable material with a use by date of approximately 3 days from opening the packaging.

### Indicator Material Formulations

Tables 1 and 2, below, exemplify suitable formulations of the indicator material for use in a self-supporting film. The formulation given in Table 1 is for a transparent composition, whilst that given in Table 2 is for an opaque composition in which the contrast material is combined with the indicator material.

TABLE 1
Example transparent composition of indicator material suitable
for use in a self-supporting film, comprising Cresol Red

| | Percentage in |
|---|---|
| Component | composition (%) |
| Water | 27.0 |
| Tetrabutylammonium hydroxide (base) | 18.5 |
| (40% in water) | |
| Cresol Red | 3.5 |
| Joncryl ® FLX 5010 (self-crosslinking | 40.0 |
| acrylic emulsion) | |
| Syloid ® 244 (hydrophilic silica) | 10.0 |
| Aerosil ® R972 (hydrophobic silica) anti- | 1.0 |
| settlement agent | |

TABLE 2
Example opaque composition comprising the indicator material suitable
for use in a self-supporting film, comprising Cresol Red

| | Percentage in |
|---|---|
| Component | composition (%) |
| Water | 18.5 |
| Tetrabutylammonium hydroxide (base) | 18.5 |
| (40% in water) | |
| Cresol Red | 3.5 |
| Joncryl ® FLX 5010 (self-crosslinking | 35.0 |
| acrylic emulsion) | |
| Syloid ® 244 (hydrophilic silica) | 9.5 |
| Titanium dioxide (whitener for opaque | 15.0 |
| indicator) | |

TABLE 3
Example transparent composition of the indicator material suitable
for use in a self-supporting film, comprising m-Cresol Purple

| | Percentage in |
|---|---|
| Component | composition (%) |
| Water | 27.0 |
| Tetrabutylammonium hydroxide (base) | 18.5 |
| (40% in water) | |
| m-Cresol Purple | 3.5 |
| Joncryl ® FLX 5010 (self-crosslinking | 40.0 |
| acrylic emulsion) | |
| Syloid ® 244 (hydrophilic silica) | 10.0 |
| Aerosil ® R972 (hydrophobic silica) anti- | 1.0 |
| settlement agent | |

TABLE 4
Example transparent composition of the indicator material suitable
for use in a self-supporting film, comprising Phenol Red

| | Percentage in |
|---|---|
| Component | composition (%) |
| Water | 27.5 |
| Tetrabutylammonium hydroxide (base) | 18.5 |
| (40% in water) | |
| Phenol Red | 3.0 |
| Joncryl ® FLX 5010 (self-crosslinking | 40.0 |
| acrylic emulsion) | |
| Syloid ® 244 (hydrophilic silica) | 10.0 |
| Aerosil ® R972 (hydrophobic silica) anti- | 1.0 |
| settlement agent | |

1. A self-supporting film comprising:
(i) a gas barrier layer;
(ii) a semi-permeable layer, wherein the semi-permeable layer comprises a polymeric material; and
(iii) an indicator material provided between the gas barrier layer and the semi-permeable layer;
wherein the indicator material is in direct contact with the gas barrier layer.

2. The self-supporting film of claim 1, wherein the gas barrier layer and semi-permeable layer are of substantially equal surface area.

3. The self-supporting film of claim 1, wherein the self-supporting film does not comprise a release layer or an outer adhesive layer.

4. The self-supporting film of claim 1, wherein the indicator material is a colourimetric indicator material and/or wherein the indicator material is capable of changing colour when exposed to a first substance, wherein the gas barrier layer is less permeable to the first substance than the semi-permeable layer.

5. (canceled)

6. The self-supporting film of claim 4, wherein the first substance is carbon dioxide.

7. The self-supporting film of claim 1,
wherein the gas barrier layer has an oxygen transmission rate from about 0.01 to about 40 cc/m²/day.

8. The self-supporting film of claim 1, wherein the semi-permeable layer has an oxygen transmission rate from about 30 to about 200 cc/m²/day.

9. The self-supporting film of claim 1, wherein the gas barrier layer has a carbon dioxide transmission rate from about 0.01 to about 20 cc/m²/day.

10. The self-supporting film of claim 1, wherein the semi-permeable layer has a carbon dioxide transmission rate from about 5 to about 200 cc/m²/day.

11. The self-supporting film of claim 1, wherein the self-supporting film further comprises at least one reference material.

12. The self-supporting film of claim 11, wherein the at least one reference material is positioned at the same depth as the indicator material, and adjacent to the indicator material.

13. The self-supporting film of claim 1, further comprising an adhesive layer.

14. The self-supporting film of claim 13, wherein the indicator material is positioned between the gas barrier layer and the adhesive layer.

15. The self-supporting film of claim 13, wherein the adhesive layer is not in contact with the indicator material.

16. The self-supporting film of claim 1, further comprising a contrast material adjacent to the indicator material, optionally on a side of the indicator material opposite the gas barrier layer.

17. The self-supporting film of claim 1, wherein the gas barrier layer comprises a coating that reduces the permeability of the gas barrier layer to a first substance and/or oxygen.

18. The self-supporting film of claim 1, wherein the gas barrier layer is transparent or semi-transparent.

19. The self-supporting film of claim 1, wherein the gas barrier layer is provided as, forms or comprises an upper or outermost layer of the self-supporting film.

20. The self-supporting film of claim 1, wherein the semi-permeable layer is provided as, forms or comprises a lower or innermost layer of the self-supporting film.

21. The self-supporting film according to claim 1, where the self-supporting film is a food packaging film.

22. An item of food packaging comprising the packaging film of claim 21.

23. (canceled)

24. A method of manufacturing a self-supporting film, the method comprising:
(i) providing a gas barrier layer;
(ii) providing a semi-permeable layer;
(iii) applying an indicator material onto the gas barrier layer or the semi-permeable layer; and
(iv) bonding and/or laminating the gas barrier layer and the semi-permeable layer;
wherein the indicator material is positioned between the gas barrier layer and the semi-permeable layer.
